(19) United States
(12) Patent Application Publication
(10) Pub. No.: US 2012/0330596 A1
(43) Pub. Date: Dec. 27, 2012

(54) SELF-CALIBRATING SENSOR, SYSTEM, AND COMPUTER PROGRAM PRODUCT

(75) Inventor: Andrian Ivanovich Kouznetsov, Santa Barbara, CA (US)

(73) Assignee: GENERAL ELECTRIC COMPANY, Schenectady, NY (US)

(21) Appl. No.: 13/164,828

(22) Filed: Jun. 21, 2011

(51) Int. Cl. G01D 18/00; G01N 33/00

(52) U.S. Cl. 702/104; 73/1.06

(57) ABSTRACT

A sensor system includes a sensor unit configured for communication has a sensor controller that initiates calibration of the sensor when a respective calibration condition occurs. The calibration condition may involve detection of appropriate environmental conditions and/or a signal received via a communication component. The sensor controller may acquire missing information with the communication. A master controller may be used that receives data from multiple sensor units and distributes information and/or sends a calibration initiation signal.

### BACKGROUND OF THE INVENTION

The disclosure relates generally to calibration of sensor hardware and software related thereto, and more particularly to self-calibrating sensing hardware and software, especially gas sensors in shipping containers.

A gas sensor, such as a carbon dioxide sensor in a food shipping container, may be subjected to a variety of factors that could cause sensor instability over time, including frequent temperature cycling, marine atmosphere, shock, vibration, and corrosive gases. As a result, such a sensor will typically require recalibration from time to time, which can be facilitated by connecting the sensor to a container controller, such as via a MODBUS network. The container controller can initiate a sensor calibration, in which the sensor's output is measured under known conditions so that, if necessary, corrections may be made in processing the output. These corrections account for sensor instability, changes in the connection between the sensor and a device using its output, and other variables.

In many circumstances, it is also advantageous for a sensor to adjust its own output, effecting a self-calibration, in response to a signal, such as a trigger from a controller, detection of calibration conditions, a switch operated by a user, or the like. However, calibration and self-calibration may only be performed when conditions around the sensor are at particular, known, and/or standard values of, for example, temperature, humidity, concentration of various gases, and other conditions as may be appropriate and/or desired. If conditions and/or time for calibration are chosen poorly, additional errors may be created in the sensing element that could cause inaccuracies in system operation. Correct identification of conditions and time for self-calibration, therefore, may be important to ensure proper and/or more efficient and/or more accurate system performance.

### BRIEF DESCRIPTION OF THE INVENTION

Embodiments of the invention disclosed herein can take the form of a self-calibrating sensor having a sensing element configured to detect a first condition and a communication component configured to enable the sensor to send and receive information. A sensor controller may be configured to communicate with the first sensing element and to monitor for a predefined calibration condition, including acquiring any needed information with the communication component. When the predefined calibration condition is detected, the sensor controller may initiate calibration of the sensing element.

Another embodiment includes a self-calibrating sensor system having a first sensor configured for communication. The sensor may include a sensing element, a communication component, and a sensor controller configured to send data from the sensing element with the communication component and to receive data with the communication component. The sensor controller may monitor for a calibration condition, including using data from the communication component, and initiate calibration of at least its respective sensing element responsive to detecting a calibration condition.

Another embodiment includes a computer program product for enabling sensor self-calibration in a sensor system, the system including a first sensor with a sensing element, a sensor controller, and a storage device configured for communication with the sensor controller and to store the computer program product. The sensor controller may include a computing device configured to execute the computer program product, the computer program product comprising instructions in the form of computer executable program code that configures the sensor controller to send data from the sensing element and to receive data from a communication component, monitor for a calibration condition, and initiate calibration of at least its respective sensing element responsive to detecting a calibration condition.

Other aspects of the invention provide methods, systems, program products, and methods of using and generating each, which include and/or implement some or all of the actions described herein. The illustrative aspects of the invention are designed to solve one or more of the problems herein described and/or one or more other problems not discussed.

### BRIEF DESCRIPTION OF THE DRAWING

These and other features of the disclosure will be more readily understood from the following detailed description of the various aspects of the invention taken in conjunction with the accompanying drawings that depict various aspects of the invention.

FIG. 1 shows a schematic diagram of a sensor unit according to embodiments disclosed herein.

FIG. 2 shows a schematic diagram of another sensor unit according to embodiments disclosed herein.

FIG. 3 shows a schematic diagram of a multiple sensor arrangement according to embodiments herein.

FIG. 4 shows a schematic diagram of another multiple sensor arrangement according to embodiments herein.

FIG. 5 shows a schematic flow diagram of a method according to embodiments of the invention.

FIG. 6 shows a schematic diagram of a climate controlled shipping/cargo container in which embodiments of the invention may be employed.

FIG. 7 shows a schematic diagram of an environment including a computer system in which embodiments of the invention may be employed.

It is noted that the drawings may not be to scale. The drawings are intended to depict only typical aspects of the invention, and therefore should not be considered as limiting the scope of the invention. In the drawings, like numbering represents like elements between the drawings.

The detailed description explains embodiments of the invention, together with advantages and features, by way of example with reference to the drawings.

### DETAILED DESCRIPTION OF THE INVENTION

As indicated above, aspects of the invention provide a system, method, and computer program product for sensor self-calibration. As used herein, “sensor calibration” means adjusting sensor output and/or interpretation thereof at a known value of a variable the sensor is configured to measure, preferably at known values of a plurality of variables, such as by a logic component of a sensor adjusting its processing and/or output based on what a sensing element sends it at the known value(s). “Self-calibration” means that a sensor calibrates its output itself. Also as used herein, unless otherwise noted, the term “set” means one or more (i.e., at least one) and the phrase “any solution” means any now known or later developed solution. Similarly, where elements are described and/or recited in the singular, it should be recognized that multiple of such elements are included unless otherwise noted. Thus, “a” generally means “at least one” throughout the instant application, including the claims.

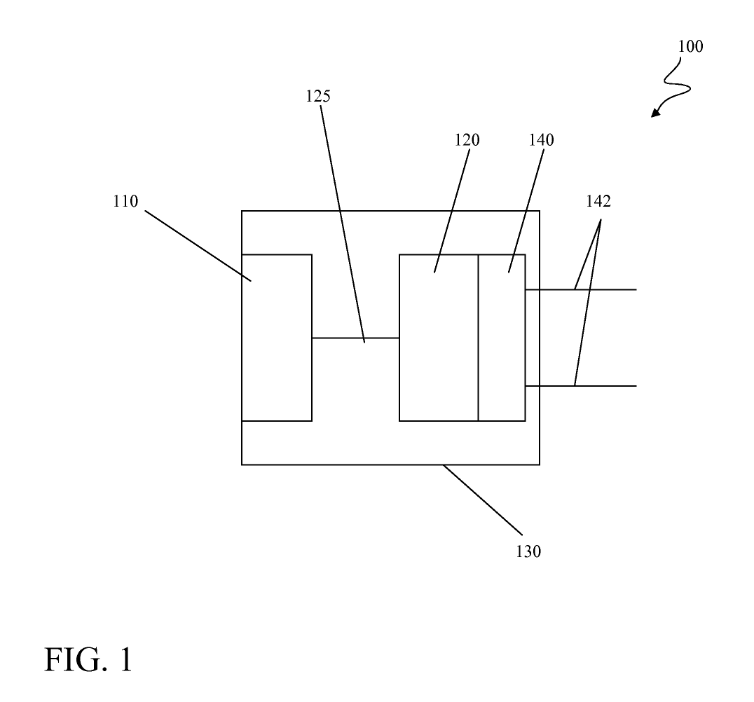

With reference to FIG. 1, embodiments of the invention disclosed herein include a self-calibrating sensor unit 100. Sensor unit 100 may include a sensing element 110 configured to detect an environmental condition, such as temperature, relative humidity, concentration of a gas, and/or any other condition as may be appropriate and/or desired. A sensor controller 120 may be connected to sensing element 110 via a connection 125 so that sensor controller 120 may monitor the environmental condition detected by sensing element 110. Sensor controller 120 may be a simple logic circuit, but may be more complex, up to and including an integrated circuit or other computing device. Sensor unit 100 in embodiments may be at least partially enclosed by a housing 130 and may include a network or communication component 140 and/or a network or communication connection 142. Sensor unit 100 may be regarded as a sensor system unto itself even as it may be part of a larger, multiple sensor arrangement described below.

While sensing element 110, sensor controller 120, and/or communication component 140 are shown as discrete elements in FIG. 1, it should be clear that this is for convenience, as an example, and/or may represent a logical distinction as opposed to a physical distinction—they could be combined on a single logic board, ASIC, or other device within the scope of embodiments. By collecting information from different inputs including, but not limited to sensing element 110 itself, other sensor units 100, and/or to a master or network controller (described below), the decision to initialize sensing element calibration could be generated by sensor unit 100 itself, a master or network controller, and/or a distributed sensor network process in which multiple sensors 100 effectively collaborate.

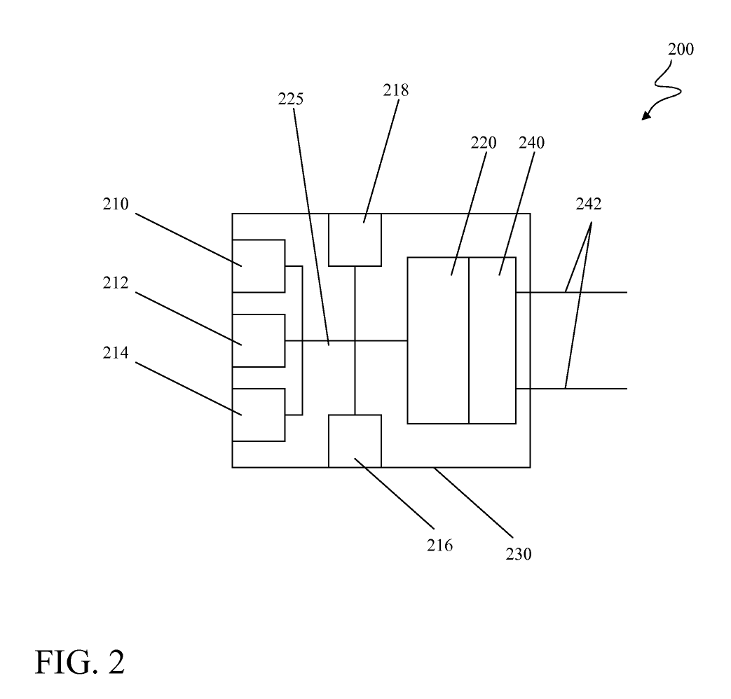

With reference to FIG. 2, a sensor unit 200 according to other embodiments may include multiple sensing elements 210, 212, 214, 216, 218 connected to a sensor controller 220. The number of sensing elements in a sensor unit may vary as appropriate and/or desired for a particular application of the sensor unit. Sensor controller 220 may monitor all sensing elements 210-218 via a connection or internal network 225. As with the example shown in FIG. 1, a sensor unit 200 with multiple sensing elements 210-218 may be at least partially enclosed by a housing 230 and may include a network or communications arrangement 240 and/or a network or communications connection 242. Sensor unit 200 may be regarded as a sensor system unto itself even as it may be part of a larger, multiple sensor arrangement described below. As with the example sensor unit 100 shown schematically in FIG. 1, while sensing element 210, sensor controller 220, and communication component 240 are shown as discrete elements in FIG. 2, this is for convenience, as an example, and/or may represent a logical distinction as opposed to a physical distinction—they could be combined on a single logic board, ASIC, or other device within the scope of embodiments. By collecting information from different inputs including, but not limited to sensing element 210 itself, other sensor units 200, and/or to a master or network controller (described below), the decision to initialize sensing element calibration could be generated by sensor unit 200 itself, a master or network controller, and/or a distributed sensor network process in which multiple sensors 100 effectively collaborate.

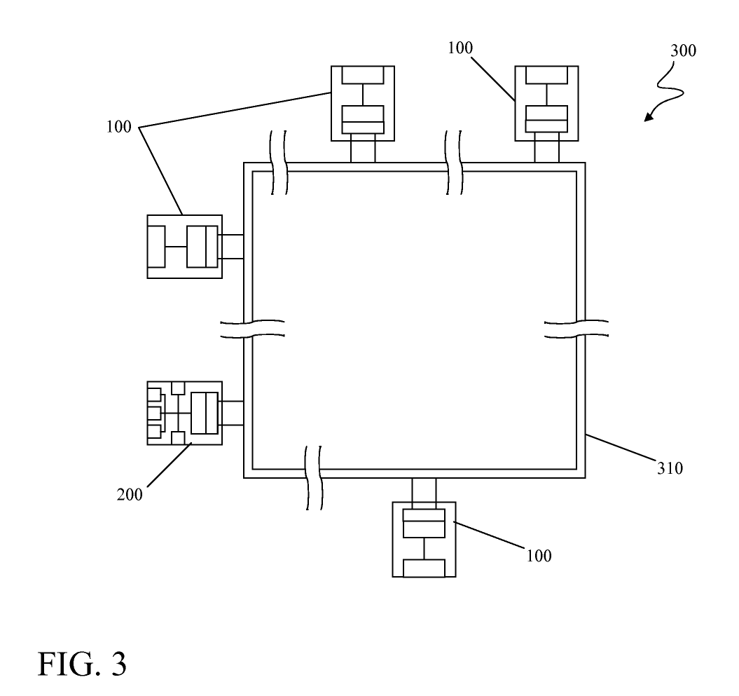

As seen in FIG. 3, embodiments may comprise a multiple sensor arrangement or sensor system 300 employing multiple sensor units 100 and/or 200 connected by a common network 310 or other communications arrangement, such as a MODBUS network, an Ethernet network, a Zigbee network, and/or any other suitable communications network. Communication between sensor units 100, 200 may include transmission/receipt of values of environmental conditions detected by the sensor units so that each sensor controller of a respective sensor unit may have access to and/or acquire all environmental condition information required to determine whether a calibration condition has been achieved. In response to a calibration condition for a particular sensing element of a particular sensor unit being achieved, the respective sensor controller initiates self-calibration of the particular sensing element.

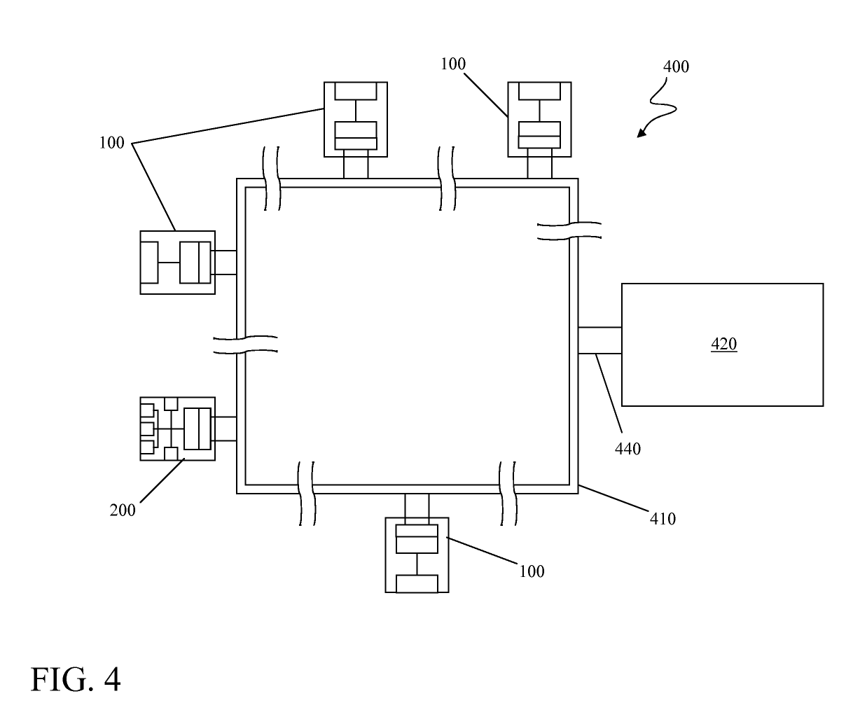

Alternatively, as seen in FIG. 4, a multiple sensor arrangement or sensor system 400 with multiple sensor units 100, 200 communicating over a common network 410 may include a master controller 420. Master controller 420 may receive information from sensor units on the network 410, such as values of environmental conditions detected, information about sensing elements included in sensor units, and/or any other information as may be appropriate and/or desired. Using information received from sensor units, master controller 420 may monitor for calibration conditions for respective sensor units and/or respective sensing elements and send a signal to the respective sensor controller when a calibration condition has been achieved, thus causing the respective sensor controller to initiate self-calibration with respect to the particular sensing element.

In operation of sensor system 300 and/or sensor system 400, output of a sensing element 110, 210 of a sensor unit 100, 200 may change in response to environmental conditions. Sensor controller 120, 220 of a sensor unit 100, 200 typically may engage in data acquisition and/or processing, acquiring and/or receiving a signal from sensing element 110, 210 and calculating a measured value based on such signal. Communication component 140, 240 may enable sensor unit 100, 200 to communicate to network 310, 410, which may include additional sensors 100, 200 and/or a master or network controller 420. Sensing element 110, 210, sensor controller 120, 220, and communication component 140, 240 may represent functions, not necessarily physical units, and some or all may be contained in a single physical unit. For example, an integrated temperature sensor may have all these components located on the same silicon die. In other embodiments, sensor controller 120, 220 and communication component 140, 240 may be functional blocks of firmware running on one computing device, such as a microcontroller, or other variations may be employed as desired and/or appropriate.

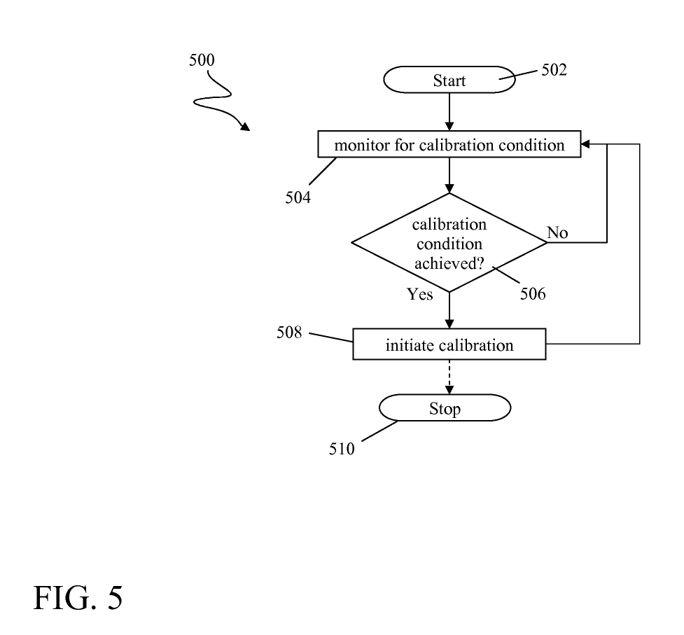

As has been suggested above, and with reference to FIG. 5, embodiments of the invention disclosed herein include and/or employ an automatic sensor self-calibration method 500. After start (block 502), the method includes monitoring for a calibration condition having been achieved (block 504), which may, in embodiments, be a signal from another controller. If a calibration condition has been achieved (block 506), then calibration is initiated (block 508). Otherwise, monitoring continues unless an instruction is given or an error causes the method to stop (block 510). The method may be performed by a sensor controller of a sensor unit in embodiments. In addition, the method may be performed by a master controller in embodiments including such a device.

In other words, embodiments of the invention include a self-calibrating sensor system 300, 400 with one or more sensors 100, 200, each sensor 100, 200 being configured for connection to and communication over a common network 310, 410. Each sensor 100, 200 may include one or more sensing elements 110, 210-218 and a sensor controller 120, 220, each sensor controller 120, 220 being configured to send data from the sensing element(s) 110, 210-218 to and to receive data from common network 310, 410. Each sensor controller 120, 220 also may monitor for a signal (block 504), such as a signal from another controller indicating that a calibration condition has been achieved.

When a calibration condition is detected by a sensor controller 120, 220, (block 506), that sensor controller 120, 220 may initiate self-calibration of at least its respective sensing element 110, 210-218, (block 508). In addition, the sensor controller 120, 220 detecting the calibration condition may broadcast a signal over the network 310, 410 to cause another sensor controller 120, 220 of a sensor 100, 200 to detect a calibration condition (block 506).

The detection of a calibration condition may include using data received from the common network 310, 410 that the respective sensor 100, 200 does not itself detect and/or collect. For example, in embodiments the sensor 100, 200 may be a carbon dioxide sensor for which the calibration condition is detection of suitable pressure, temperature, and/or relative humidity. If the carbon dioxide sensor does not include sensing elements that provide information necessary to determine that calibration conditions have been reached, the sensor controller of the carbon dioxide sensor may use information from the common network. For example, if the carbon dioxide sensor includes a temperature sensing element, but does not include a relative humidity sensing element and/or pressure sensing element, the sensor controller may collect the missing information from the common network as supplied by another sensor(s) that includes the missing sensing element(s). This may be achieved without a master controller, instead relying on communication between sensor unit sensor controllers over the network.

However, as shown in FIG. 4, a master controller 420 may be used to collect information from common network 410 and determine when calibration conditions have been reached for each sensor 100, 200 on common network 410. When calibration conditions have been reached for a sensor 100, 200, master controller 420 may send a signal over common network 410 that a respective sensor controller 120, 220 may use to initiate self-calibration of that sensor (block 508 of FIG. 5). In such an embodiment, the sensor controller 120, 220 of each sensor 100, 200 may be less complex since master controller 420 does work that would be done by a sensor controller 120, 220 in a system without a master controller 420.

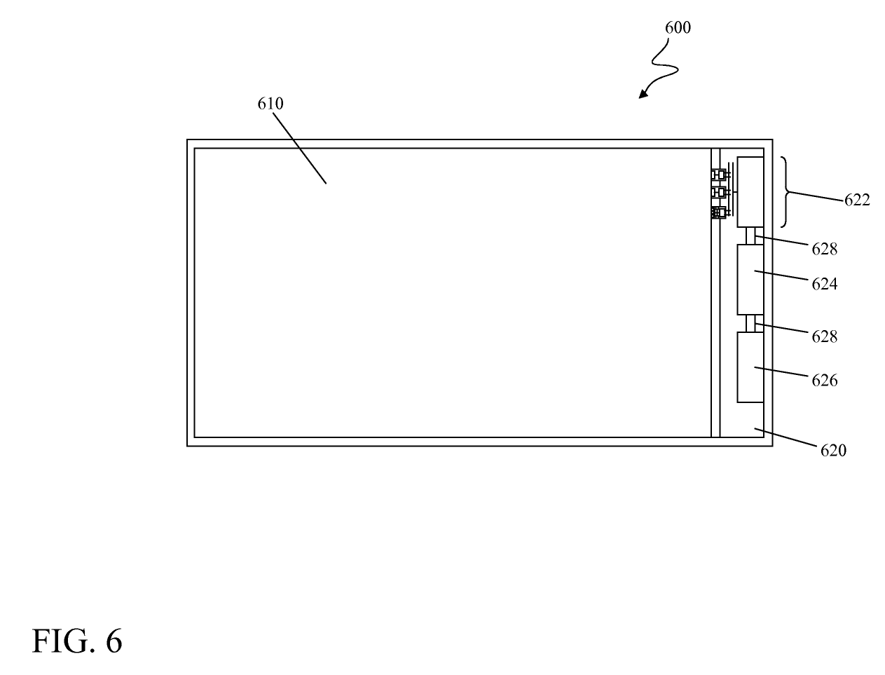

In a particular implementation, as seen in FIG. 6, a climate controlled shipping or cargo container 600 may include a cargo bay 610 and a climate control bay 620. Climate control bay may include a self-calibrating sensor system 622 including one or more sensors deployed in shipping container 600 and connected to climate control units, such as a first climate control unit 624 and/or a second climate control unit 626. For example, a control unit may include a temperature control unit, a gas mixture control unit, a relative humidity control unit, and or control units for other conditions as may be desired and/or appropriate. Sensor system 622 may be connected to a control unit via a communications arrangement, such as a common network, in shipping container 600. For example, shipping container 600 may include a MODBUS network.

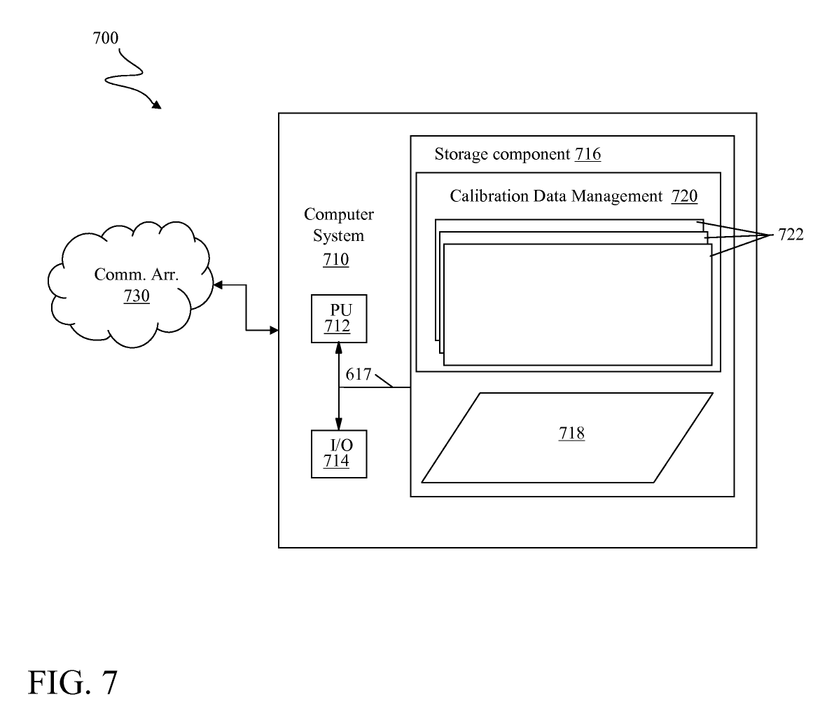

Turning to FIG. 7, an illustrative environment 700 for an automatic sensor self-calibration computer program product is schematically illustrated according to an embodiment of the invention. To this extent, environment 700 includes a computer system 710, such as a sensor controller 120, 220, a master controller 420, or other computing device that may be part of a sensor unit 100, 200 and/or a sensor system 300, 400, that may perform a process described herein in order to execute an automatic sensor self-calibration method according to embodiments. In particular, computer system 710 is shown including a sensor unit or system calibration program 720, which makes computer system 710 operable to manage data in a sensor unit or system by performing a process described herein, such as an embodiment of the sensor unit or system calibration method discussed above.

Computer system 710 is shown including a processing component or unit (PU) 712 (e.g., one or more processors), an input/output (I/O) component 714 (e.g., one or more I/O interfaces and/or devices), a storage component 716 (e.g., a storage hierarchy), and a communications pathway 717. In general, processing component 712 executes program code, such as sensor unit or system calibration program 720, which is at least partially fixed in storage component 716, which may include one or more computer readable storage medium or device. While executing program code, processing component 712 may process data, which may result in reading and/or writing transformed data from/to storage component 716 and/or I/O component 714 for further processing. Pathway 717 provides a communications link between each of the components in computer system 710. I/O component 714 may comprise one or more human I/O devices, which enable a human user to interact with computer system 710 and/or one or more communications devices to enable a system user to communicate with computer system 710 using any type of communications link. In embodiments, a communications arrangement 730, such as networking hardware/software, enables computing device 710 to communicate with other devices in and outside of a node in which it is installed. To this extent, sensor unit or system calibration program 720 may manage a set of interfaces (e.g., graphical user interface(s), application program interface, and/or the like) that enable human and/or system users to interact with sensor unit or system calibration program 720. Further, sensor unit or system calibration program 720 may manage (e.g., store, retrieve, create, manipulate, organize, present, etc.) data, such as sensor unit or system data 718, using any solution.

Computer system 710 may comprise one or more general purpose computing articles of manufacture (e.g., computing devices) capable of executing program code, such as sensor unit or system calibration program 720, installed thereon. As used herein, it is understood that “program code” means any collection of instructions, in any language, code or notation, that cause a computing device having an information processing capability to perform a particular action either directly or after any combination of the following: (a) conversion to another language, code or notation; (b) reproduction in a different material form; and/or (c) decompression. Additionally, computer code may include object code, source code, and/or executable code, and may form part of a computer program product when on at least one computer readable medium. It is understood that the term “computer readable medium” may comprise one or more of any type of tangible medium of expression, now known or later developed, from which a copy of the program code may be perceived, reproduced, or otherwise communicated by a computing device. For example, the computer readable medium may comprise: one or more portable storage articles of manufacture; one or more memory/storage components of a computing device; paper; and/or the like. Examples of memory/storage components include magnetic media (floppy diskettes, hard disc drives, tape, etc.), optical media (compact discs, digital versatile/video discs, magneto-optical discs, etc.), random access memory (RAM), read only memory (ROM), flash ROM, erasable programmable read only memory (EPROM), or any other computer readable storage medium now known and/or later developed and/or discovered on which the computer program code is stored and with which the computer program code can be loaded into and executed by a computer. When the computer executes the computer program code, it becomes an apparatus for practicing the invention, and on a general purpose microprocessor, specific logic circuits are created by configuration of the microprocessor with computer code segments. A technical effect of the executable instructions is to implement an automatic sensor self-calibration method and/or system and/or computer program product that initiates a self-calibration of a sensing element or sensor unit when a calibration condition is achieved and/or detected and/or occurs. Detecting a calibration condition may include detection of a predefined threshold value of an environmental condition, a predefined range of values of an environmental condition, a signal from another controller, and/or other criteria as may be desired and/or appropriate.

The computer program code may be written in computer instructions executable by the controller, such as in the form of software encoded in any programming language. Examples of suitable computer instruction and/or programming languages include, but are not limited to, assembly language, Verilog, Verilog HDL (Verilog Hardware Description Language), Very High Speed IC Hardware Description Language (VHSIC HDL or VHDL), FORTRAN (Formula Translation), C, C++, C#, Java, ALGOL (Algorithmic Language), BASIC (Beginner All-Purpose Symbolic Instruction Code), APL (A Programming Language), ActiveX, Python, Perl, php, Tcl (Tool Command Language), HTML (HyperText Markup Language), XML (eXtensible Markup Language), and any combination or derivative of one or more of these and/or others now known and/or later developed and/or discovered. To this extent, sensor unit or system calibration program 720 may be embodied as any combination of system software and/or application software.

Further, sensor unit or system calibration program 720 may be implemented using a set of modules 722. In this case, a module 722 may enable computer system 710 to perform a set of tasks used by sensor unit or system calibration program 720, and may be separately developed and/or implemented apart from other portions of sensor unit or system calibration program 720. As used herein, the term “component” means any configuration of hardware, with or without software, which implements the functionality described in conjunction therewith using any solution, while the term “module” means program code that enables a computer system 710 to implement the actions described in conjunction therewith using any solution. When fixed in a storage component 716 of a computer system 710 that includes a processing component 712, a module is a substantial portion of a component that implements the actions. Regardless, it is understood that two or more components, modules, and/or systems may share some/all of their respective hardware and/or software. Further, it is understood that some of the functionality discussed herein may not be implemented or additional functionality may be included as part of computer system 710.

When computer system 710 comprises multiple computing devices, each computing device may have only a portion of sensor unit or system calibration program 720 fixed thereon (e.g., one or more modules 722). However, it is understood that computer system 710 and sensor unit or system calibration program 720 are only representative of various possible equivalent computer systems that may perform a process described herein. To this extent, in other embodiments, the functionality provided by computer system 710 and sensor unit or system calibration program 720 may be at least partially implemented by one or more computing devices that include any combination of general and/or specific purpose hardware with or without program code. In each embodiment, the hardware and program code, if included, may be created using standard engineering and programming techniques, respectively.

Regardless, when computer system 710 includes multiple computing devices, the computing devices may communicate over any type of communications link. Further, while performing a process described herein, computer system 710 may communicate with one or more other computer systems using any type of communications link. In either case, the communications link may comprise any combination of various types of wired and/or wireless links; comprise any combination of one or more types of networks; and/or utilize any combination of various types of transmission techniques and protocols now known and/or later developed and/or discovered.

As discussed herein, sensor unit or system calibration program 720 enables computer system 710 to implement an automatic sensor self-calibration product and/or method, such as that shown schematically in FIG. 5. Computer system 710 may obtain sensor unit or system data 718 using any solution. For example, computer system 710 may generate and/or be used to generate sensor unit or system data 718, retrieve sensor unit or system data 718 from one or more data stores, receive sensor unit or system data 718 from another system or device in or outside of a sensor unit, sensor system, and/or the like.

In another embodiment, the invention provides a method of providing a copy of program code, such as sensor unit or system calibration program 720 (FIG. 7), which implements some or all of a process described herein, such as that shown schematically in and described with reference to FIG. 5. In this case, a computer system may process a copy of program code that implements some or all of a process described herein to generate and transmit, for reception at a second, distinct location, a set of data signals that has one or more of its characteristics set and/or changed in such a manner as to encode a copy of the program code in the set of data signals. Similarly, an embodiment of the invention provides a method of acquiring a copy of program code that implements some or all of a process described herein, which includes a computer system receiving the set of data signals described herein, and translating the set of data signals into a copy of the computer program fixed in at least one computer readable medium. In either case, the set of data signals may be transmitted/received using any type of communications link.

In still another embodiment, the invention provides a method of generating a system for implementing an automatic sensor self-calibration product and/or method. In this case, a computer system, such as computer system 710 (FIG. 7), can be obtained (e.g., created, maintained, made available, etc.), and one or more components for performing a process described herein can be obtained (e.g., created, purchased, used, modified, etc.) and deployed to the computer system. To this extent, the deployment may comprise one or more of: (1) installing program code on a computing device; (2) adding one or more computing and/or I/O devices to the computer system; (3) incorporating and/or modifying the computer system to enable it to perform a process described herein; and/or the like.

It is understood that aspects of the invention can be implemented as part of a business method that performs a process described herein on a subscription, advertising, and/or fee basis. That is, a service provider could offer to implement an automatic sensor self-calibration product and/or method as described herein. In this case, the service provider can manage (e.g., create, maintain, support, etc.) a computer system, such as computer system 710 (FIG. 7), that performs a process described herein for one or more customers. In return, the service provider can receive payment from the customer(s) under a subscription and/or fee agreement, receive payment from the sale of advertising to one or more third parties, and/or the like.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A self-calibrating sensor comprising:
a first sensing element configured to detect a first condition;
a communication component configured to enable the sensor to send and receive information; and
a controller configured to communicate with the first sensing element, to monitor for a predefined calibration condition, including acquiring any needed information with the communication component, and to initiate calibration of the sensor to output of the sensing element responsive to the predefined calibration condition.

2. The sensor of claim 1, wherein the predefined calibration condition includes detection of a value of a second environmental condition within a predefined range of values of the second environmental condition.

3. The sensor of claim 2, further comprising at least a second sensing element for at least the second environmental condition, each sensing element being configured to communicate with the controller.

4. The sensor of claim 2, wherein the controller is configured to acquire the second environmental condition value via the communication component.

5. The sensor of claim 4, wherein the second environmental condition value is sent by a device connected to the network.

6. The sensor of claim 4, wherein the device includes a master controller.

7. The sensor of claim 1, wherein the calibration condition is detected by receiving a signal from a master controller.

8. The sensor of claim 1, wherein the calibration condition is detected by receiving a signal from a user-operated switch.

9. A self-calibrating sensor system comprising:
a first sensor configured including:
a sensing element configured to detect a first condition;
a communication component; and
a controller configured to:
send data from the sensing element with the communication component and to receive data with the communication component;
monitor for a calibration condition, including using data from the communication component; and
initiate calibration of the sensing element responsive to detecting a calibration condition.

10. The system of claim 9, wherein detection of a calibration condition includes receiving a value of a second condition from the communication component.

11. The system of claim 10, wherein the value of the second condition is sent by another sensor.

12. The system of claim 9, wherein the monitoring for a calibration condition includes monitoring for a signal sent by a master controller.

13. The system of claim 12, wherein the master controller is configured to receive data from a plurality of sensors.

14. The system of claim 12, wherein the master controller is part of a climate control system of a shipping container.

15. A computer program product for enabling sensor calibration in a sensor system, the system including a first sensor with a sensing element, a sensor controller, and a storage device configured for communication with the sensor controller and to store the computer program product, the sensor controller including a computing device configured to execute the computer program product, the computer program product comprising instructions in the form of computer executable program code that configures the sensor controller to:
send data from the sensing element and receive data with a communication component;
monitor for a calibration condition; and
initiate calibration of the sensing element responsive to detecting the calibration condition.

16. The computer program product of claim 15, wherein the calibration condition includes detection of a value of a first condition in a predefined range of values of the first condition.

17. The computer program product of claim 15, wherein the controller is further configured by the computer executable program code to detect a calibration condition using data received from the communication component.

18. The computer program product of claim 17, wherein the data received from the communication component is sent by another sensor.

19. The computer program product of claim 17, wherein the calibration condition is detected by receiving a signal sent by a master controller.

20. The computer program product of claim 19, wherein the master controller is configured to receive data from a plurality of sensors.
